(19) United States
(12) Patent Application Publication
(10) Pub. No.: US 2008/0145400 A1
(43) Pub. Date: Jun. 19, 2008

(54) Ion Bombardment of Medical Devices

(76) Inventors: Jan Weber, Maastricht (NL); Natalia Shevchenko, Dresden (DE)

Correspondence Address: FISH & RICHARDSON PC, P.O. BOX 1022, MINNEAPOLIS, MN, 55440-1022, US

(21) Appl. No.: 11/934,415

(22) Filed: Nov. 2, 2007

Related U.S. Application Data
(60) Provisional application No. 60/856,583, filed on Nov. 3, 2006.
(60) Provisional application No. 60/875,122, filed on Dec. 15, 2006.

(51) Int. Cl. A61F 2/28; A61C 13/00; A61M 29/00; C23C 8/36

(52) U.S. Cl. 424/423; 433/201.1; 623/16.11; 606/200; 148/239

(57) ABSTRACT

A medical device can include a metal member including a porous first portion with pores extending from a surface of the metal member into the first portion and non-porous second portion. The first portion can have a porosity that varies with distance from the surface of the metal member.

### CROSS REFERENCE TO RELATED APPLICATIONS

This application claims the benefit of U.S. Provisional Application No. 60/856,583, filed on Nov. 3, 2006, and U.S. Provisional Application No. 60/875,122, filed on Dec. 15, 2006, both of which are incorporated herein by reference in their entireties.

### FIELD OF THE INVENTION

The invention relates to medical devices and the manufacture thereof.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, a passageway can be reopened or reinforced, or even replaced, with a medical endoprosthesis. An endoprosthesis is typically a tubular member that is placed in a lumen in the body. Examples of endoprostheses include stents, stent-grafts, and covered stents.

An endoprosthesis can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded (e.g., elastically or through a material phase transition). During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

To support a passageway and keep the passageway open, endoprostheses are sometimes made of relatively strong materials, such as stainless steel or Nitinol (a nickel-titanium alloy), formed into struts or wires.

In some cases, endoprostheses are used as a delivery mechanism for therapeutic agents.

### SUMMARY

Ion implantation of noble gases in metal substrates can provide an approach to forming medical devices (e.g., endoprostheses, dental implants, and bone implants) with pores extending from at least one surface of the medical devices. The characteristics (e.g., size, distribution, and degree of interconnection) of the pores can be controlled by varying the ion implantation parameters. For example, metal-based drug-eluting endoprostheses can be formed with a multi-layer pore system on their lumenal surfaces. A surface layer of small pores can connect a deeper layer of larger pores to the surface of the endoprostheses and control the rate of elution of therapeutic agents stored in the deeper layer of larger pores. Such metal-based endoprostheses are thought to be more bio-compatible than comparable polymeric endoprostheses. In another example, coated endoprostheses can be formed with a surface layer of pores on the endoprostheses providing attachment points for a coating (e.g., a ceramic or polymeric layer).

In one general aspect, endoprostheses include: a metal member including a porous first portion with pores extending from a surface of the metal member into the first portion and non-porous second portion; wherein the first portion has a porosity that varies with distance from the surface of the metal member.

In another general aspect, medical devices include: a metal member including a porous first portion with pores extending from a surface of the metal member into the first portion and non-porous second portion; wherein the first portion has a porosity that varies with distance from the surface of the metal member.

In another general aspect, methods of forming an endoprosthesis include: forming a pre-endoprosthesis from a metal; and forming pores in the metal by implanting ions of a noble gas in the metal.

Embodiments of these aspects can include one or more of the following features.

In some embodiments, the porosity of first portion increases with distance from the surface. In some cases, the first portion includes a surface layer of pores with a first representative pore size and an interior layer of pores with a second representative pore size that is greater than the first representative pore size, pores of the surface layer interconnected to provide a plurality of fluid flow paths extending between the surface and the interior layer. Endoprostheses can also include a therapeutic agent disposed within the interior layer of pores. In some instances, the first representative pore size is between about 0.5 and 5 nanometers (e.g., between about 1.5 and 3 nanometers). In some instances, the second representative pore size is between about 50 nanometers and 500 nanometers (e.g., between about 100 and 300 nanometers). Endoprostheses can also include a plug disposed in a bore extending between the surface and the interior layer.

In some embodiments, the metal member is a tubular member having an axis and the first portion is disposed between the second portion and the axis.

In some embodiments, the porous first portion and the non-porous second portion are integrally formed.

In some embodiments, wherein the metal member comprises struts interconnected at junctions and the pores are not present at the junctions.

In some embodiments, endoprostheses also include a coating, the coating covering a portion of the surface of the metal member and extending into the pores of the first portion. In some cases, the coating comprises a polymer. In some cases, the coating comprises a ceramic.

In some embodiments, the porosity of first portion increases with distance from the surface. In some cases, the first portion includes a surface layer of pores with a first representative pore size and an interior layer of pores with a second representative pore size that is greater than the first representative pore size, pores of the surface layer interconnected to provide a plurality of fluid flow paths extending between the surface and the interior layer. Some medical devices can also include a therapeutic agent disposed within the interior layer of pores. Some medical devices also include a plug filling a bore extending between the surface and the interior layer.

In some embodiments, medical devices also include a coating covering a portion of the surface of the metal member and extending into the pores of the first portion.

In some embodiments, the medical device forms at least part of a dental implant. In some cases, the first portion includes a surface layer of pores with a first representative pore size and the first representative pore size is less than about 200 nanometers.

In some embodiments, the medical device forms at least part of a bone implant.

In some embodiments, the medical device forms at least part of an embolic coil.

In some embodiments, forming the endoprosthesis takes place before forming the pores. In other embodiments, forming the pores takes place before forming the endoprosthesis.

In some embodiments, the noble gas is selected from the group consisting of argon and helium. In some embodiments, the metal is selected from the group consisting of titanium, stainless steel, stainless steel alloy, tungsten, tantalum, niobium, and zirconium.

In some embodiments, methods also include covering portions of the metal with a sacrificial material which limits ion implantation. In some cases, methods also include removing the sacrificial layer.

In some embodiments, implanting the ions comprises applying the ions at an implantation energy of between about 10 kiloelectron volts and 1 megaelectron volts. In some embodiments, implanting the ions comprises applying the ions at a dose of between about 15×10¹⁷ and 50×10¹⁸ ions per square centimeter.

In some embodiments, forming the pores comprises forming a surface layer of pores with a first representative pore size and an interior layer of pores with a second representative pore size that is greater than the first representative pore size, pores of the surface layer interconnected to provide a plurality of fluid flow paths extending between a surface of the metal and the interior layer of pores. In some cases, methods also include: forming a bore extending from the surface of the metal to the interior layer of pores; loading a therapeutic agent into the interior layer of pores; and placing a seal material in the bore.

In some embodiments, methods also include applying a mask to control locations at which pores are formed in the metal.

The “porosity” of an object or a portion of an object containing pores is the ratio of pore volume to total volume of the object or the portion of the object. The porosity is independent of whether the pores are empty or filled (partially or completely) with a material different than the material of the object. The pores can be isolated or interconnected voids within the object. The porosity can be measured by N2-porosimetry BET or by positronium annihilation lifetime spectroscopy (PALS).

Pore size is characterized by the length of the average perimeter of cross-sections of a pore. For a longitudinally extending pore, the relevant cross-sections can be transverse cross-sections taken across a longitudinally extending axis of the pore. A representative pore size of an object or a portion of an object represents a mean size of the pores contained in the object or portion of the object determined based on averaging the cross-sections of pores observed (e.g. as is reflected by the effect on the half-life time of the positronium within a PALS measurement)

A “non-porous” object or portion of an object is an object or portion of an object without pores measurable by PALS.

The methods and devices described herein can provide one or more advantages. By controlling ion implantation parameters, medical devices can be manufactured with porous regions whose porosity varies with distance from a surface of the medical device. In some embodiments, a highly porous interior region of the medical devices can be used to store a substance (e.g., therapeutic agent or a radioactive substance) which is gradually transferred to the surface of the medical devices through a less porous region of the medical devices. The rate of this transfer can be controlled, at least in part, by the size of the pores in the less porous region which connect pores in the more porous region to the surface of the medical device. In some embodiments, pores in communication with the surface of the medical devices can provide high surface area attachment points for coatings applied to the medical devices.

In endoprostheses with porous regions formed by ion implantation, material of the endoprostheses in the porous region is an integral part of the material of the non-porous regions of the endoprostheses. This unity of structure contrasts with the structure of endoprostheses where a porous region is formed and/or attached (e.g., by sintering) to the underlying non-porous region and can provide desirable structural stability. In addition, this can limit biocompatibility issues that can otherwise arise if the underlying substrate would be exposed for some reason because the surface region is identical in composition to the substrate (i.e., it is the substrate).

The details of one or more embodiments of the invention are set forth in the accompanying drawings and the description below. Other aspects, features, and advantages of the invention will be apparent from the description and drawings, and from the claims.

### DESCRIPTION OF DRAWINGS

FIG. 1A is a perspective view of an embodiment of an endoprosthesis.

FIG. 1B is a schematic cross-section of the endoprosthesis of FIG. 1A taken along line 1B.

FIGS. 2A and 2B are, respectively, schematic cross-sectional and plan views of an embodiment of a plasma ion implantation system.

FIG. 3 is an illustration of an embodiment of a method of making an endoprosthesis.

FIG. 4A is a perspective view of an embodiment of an endoprosthesis and FIG. 4B is an enlarged perspective view of a portion of the endoprosthesis of FIG. 4A.

FIG. 5A is a schematic cross-sectional view of an embodiment of an endoprosthesis. FIG. 5B is an enlarged cross-sectional view of a portion of the endoprosthesis of FIG. 5A.

FIGS. 6A and 6B are scanning electron micrographs of pores formed by noble gas ion implantation taken at 10,000 and 50,000 magnifications, respectively.

Like reference symbols in the various drawings indicate like elements.

### DETAILED DESCRIPTION

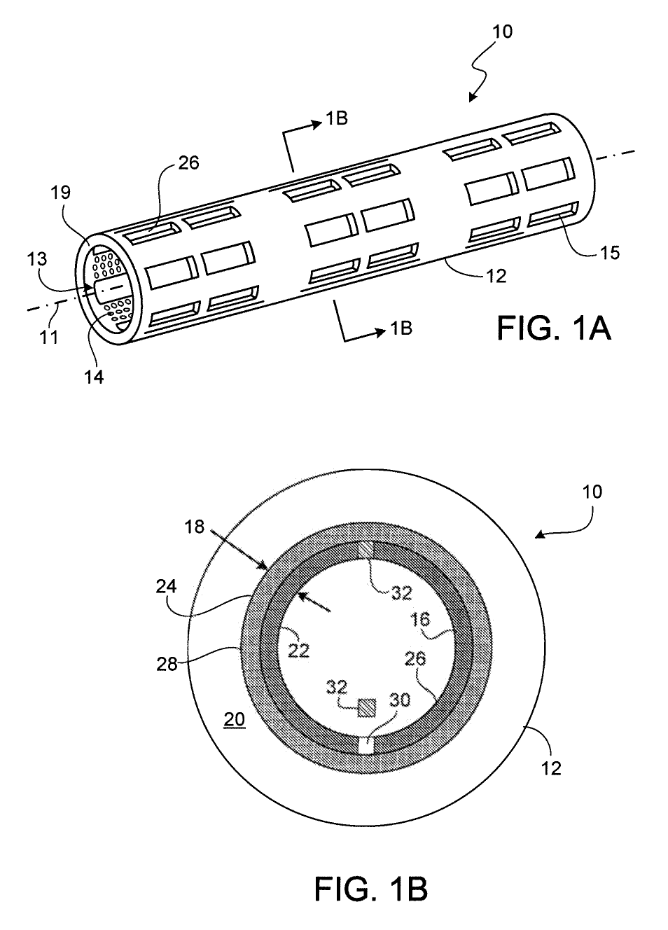

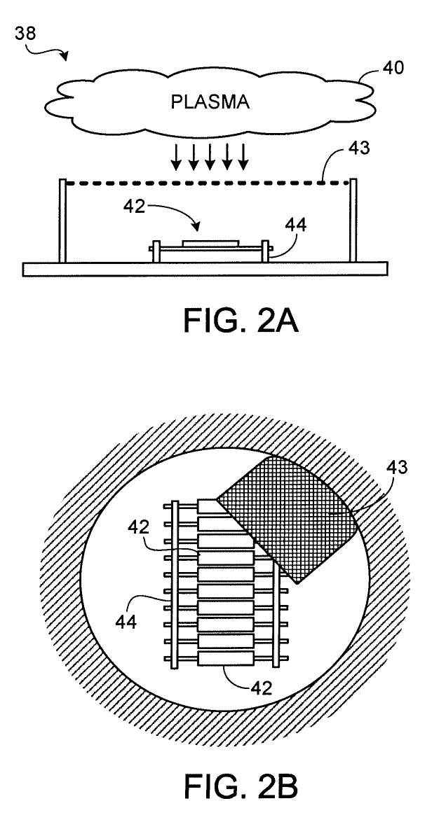

Referring to FIGS. 1A and 1B, an endoprosthesis 10 includes (e.g., comprises or consists of) a tubular metal member 12 with an axis 11. As shown, metal member 12 includes apertures 13, with aperture surfaces 15, extending through the metal member from inner or lumenal surface 16 to exterior surface 17. End surfaces 19, disposed at the ends of endoprosthesis 10, also extend from inner surface 16 to exterior surface 17.

Metal member 12 includes a porous section 18 which has a porosity that varies with distance from surface 16 (e.g., increases or decreases with distance from the surface) of metal member 12 and a non-porous section 20. Pores 14 can form an open pore system (in which different pores 14 are interconnected) or a closed pore system (in which different pores 14 are not interconnected). In certain embodiments, some pores 14 can be interconnected and/or other pores 14 may not be interconnected. Pores 14 can have an irregular cross-sectional shape or, in some embodiments, the pores can have one or more other cross-sectional shapes. For example, a pore in a metal matrix can be circular, oval (e.g., elliptical), and/or polygonal (e.g., triangular, square) in cross-section. In this embodiment, pores 14 extend from inner surface 16 of metal member 12 into the metal member. Porous section 18 includes a surface layer 22 of first pores 26 with a first representative pore size and an interior layer 24 of second pores 28 with a second representative pore size that is greater than the first representative pore size. At least some of first pores 26 of surface layer 22 are interconnected and provide a plurality of fluid flow paths extending between surface 16 and interior layer 24. The fluid flow paths are not specifically shown in FIG. 1B. The difference between open and closed pores can be detected using PALS.

In some embodiments, at least one bore 30 extends from inner surface 16 through surface layer 22 towards (e.g., to or into) interior layer 24 as shown in FIG. 1B. Bore or bores 30 provide a channel for rapidly loading second pores 28 of interior layer 24 with a therapeutic agent or other appropriate substance. For example, a nanopowder of short-life decay time isotopes (e.g., Iodine-131 or Iridium-192) could be loaded into the pores. After loading, plugs 32 can be inserted (e.g., press-fit) into bores 30 to limit the flow of such loaded therapeutic agents out of second pores 28 through the bores. Thus, bores 30 and plugs 32 can provide a mechanism for loading therapeutic agents into second pores 28 such that the therapeutic agents are then available for elution from endoprosthesis 10 through first pores 26. In some embodiments, plugs 32 can include (e.g., be made of) erodible material (e.g., large glucose molecules such as beta-cyclodextrin) which can provide an initial slow release through the first pores 26 until opening of the bores 30 due erosion of the plugs releases the remaining drug.

Examples of therapeutic agents include non-genetic therapeutic agents, genetic therapeutic agents, vectors for delivery of genetic therapeutic agents, cells, and therapeutic agents identified as candidates for vascular treatment regimens, for example, as agents targeting restenosis. In some embodiments, one or more therapeutic agents that are used in a medical device such as an endoprosthesis can be dried (e.g., lyophilized) prior to use, and can become reconstituted once the medical device has been delivered into the body of a subject. A dry therapeutic agent may be relatively unlikely to come out of a medical device (e.g., an endoprosthesis) prematurely, such as when the medical device is in storage. Therapeutic agents are described, for example, in Weber, U.S. Patent Application Publication No. US 2005/0261760 A1, published on Nov. 24, 2005, and entitled “Medical Devices and Methods of Making the Same”, and in Colen et al., U.S. Patent Application Publication No. US 2005/0192657 A1, published on Sep. 1, 2005, and entitled “Medical Devices”.

In some embodiments, endoprostheses can be configured, as shown, with first pores 26 of surface layer 22 open only to lumenal surface 16. Such endoprostheses can provide a high degree of control over the discharge rate of substances from the interior layer as the fluid mechanics of flow through the first pores can govern the discharge rate.

In some embodiments, endoprostheses can be configured with first pores 26 of surface layer 22 and/or second pores 28 of interior layer also open to aperture surfaces 15 and/or end surfaces 19. For example, ion implantation can be used to form pores 26/28 extending into a pre-endoprosthesis that are uniformly distributed across a surface of the endoprosthesis. Thus, when apertures 13 are formed (e.g., by laser cutting), some of second pores 28 can directly open onto aperture surfaces 15 as well as being connected to interior surface 16 through first pores 26. The reduction of flow control may be proportional to the ratio of the flow area of openings directly from second pores 28 to the flow area of openings of the first pores 26. In endoprostheses where this ratio is small (e.g., endoprostheses with few apertures and a large lumenal area with pores), the reduction of flow control may be negligible.

In some embodiments, first pores 26 and second pores 28 can be configured (e.g., sized and distributed) to provide a highly porous interior layer 24 to store a therapeutic agent which is gradually transferred to surface through the smaller first pores of surface layer 22. For example, the surface layer can have a first representative pore size between about 0.5 and 5 nanometers (e.g., more than about 1 nanometer, more than about 2 nanometer, more than about 3 nanometer, more than about 4 nanometer or less than about 4 nanometer, less than about 3 nanometer, less than about 2 nanometer) and the interior layer can have a second representative pore size between about 100 nanometers and 200 nanometers (e.g., between about 125 and 175 nanometers or between about 135 and 165 nanometers). The rate of this transfer is controlled, at least in part, by the size and distribution (e.g., the degree of connectivity and the tortuosity of the flow paths formed by connected pores) of the pores in the surface layer which connect pores in the interior layer to the surface of the medical device. The rate of transfer and appropriate pore size is also dependent on the size of the therapeutic molecule. If the top-layer porosity is too large, one could always partially close the first pores 26 (e.g., by chemical vapor deposition (CVD), physical vapor deposition (PVD), or pulsed laser deposition utilizing the same target material as the substrate is made of).

In some embodiments, pores 14 can be formed by implanting ions of noble gases (e.g., helium, neon, argon, krypton, xenon, and radon) in a metal portion of a pre-endoprosthesis. In one example, ion bombardment was used to implant argon ions into heated stainless steel. The implanted argon ions initially precipitated out of the stainless steel to form high concentrations of gas bubbles of uniform size with bubbles initially nucleating to form a random array. With increasing doses of argon ions, adjacent bubbles began to coalesce and, at high enough doses, form interconnected pores in the stainless steel and/or blisters on the surface of the stainless steel.

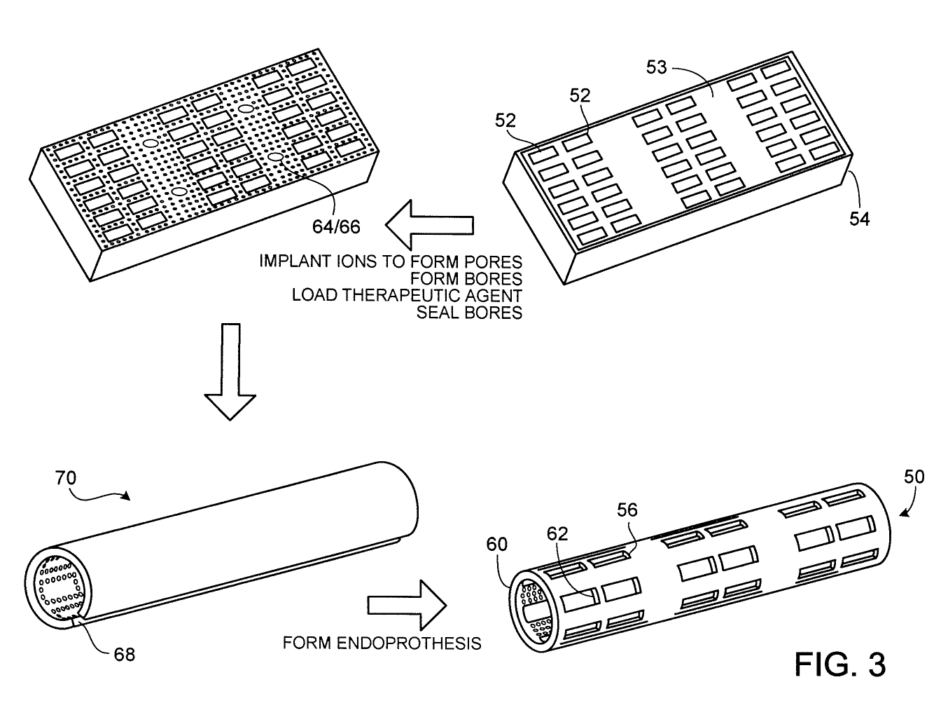

For example, referring to FIGS. 2A and 2B, a plasma ion implantation system 38 can be used to accelerate charged species (e.g., helium or argon ions in a plasma 40) at high velocity towards pre-endoprostheses 42, which are positioned on a sample holder 44. Acceleration of the charged species of plasma 40 towards pre-endoprostheses 42 is driven by an electrical potential difference between the plasma and an electrode under the pre-endoprostheses. In some embodiments, metallic endoprostheses themselves can be used as the electrode. Upon impact with an pre-endoprosthesis 42, the charged species penetrate a distance into the pre-endoprostheses due to the high ion energy, thus forming the bubbles and pores as discussed above. Generally, the penetration depth is controlled, at least in part, by the potential difference between plasma 40 and the electrode under the pre-endoprostheses 42. If desired, an additional electrode, e.g., in the form of a metal grid 43 positioned above sample holder 44, can be utilized. Such a metal grid can be advantageous to prevent direct contact of the endoprostheses with the rf-plama between high-voltage pulses and can reduce charging effects of the pre-endoprosthesis material. Plasma ion implantation has been described by Chu, U.S. Pat. No. 6,120,660; Brukner, Surface and Coatings Technology, 103-104, 227-230 (1998); and Kutsenko, Acta Materialia, 52, 4329-4335 (2004), the entire disclosure of each of which is hereby incorporated by reference herein.

Ion penetration depth and ion concentration and, thus, bubble/pore size and distribution, can be modified by changing the configuration of plasma ion implantation system 38 as well as parameters such as, for example, the type of ion, the substrate atoms, and the temperature of the substrate. For example, when the ions have a relatively low energy, e.g., 10,000 electron volts or less, penetration depth is relatively shallow (e.g., less than about 20 nanometers) when compared with increased penetration depths (e.g., up to 1 micrometers or up to 5 micrometers) when the ions have a relatively high energy, e.g., greater than 40,000 electron volts. The dose of ions being applied to a surface can range from about 1×10¹⁵ ions/cm² to about 1×10¹⁹ ions/cm², e.g., from about 5×10¹⁷ ions/cm² to about 5×10¹⁸ ions/cm². As discussed above, higher doses of ions being applied can provide larger bubbles and increased connectivity. In systems with a metal grid, the angle of incidence of the ions upon the surface of a pre-endoprosthesis can be increased thus increasing the width of a layer of bubbles/pores of the given size. For example, angles of incidence can range from approximately 90 degrees to provide a narrow layer to approximately 45 degrees to provide a wider layer.

Masking techniques can be used to control the location of pores on an endoprosthesis. In some embodiments, a blocking material (e.g., metals, ceramics, or hard polymers) can be positioned between the plasma source and a pre-endoprosthesis in which ions are being implanted without attaching the blocking material to the endoprosthesis. In some embodiments, sacrificial materials can be applied to coat portions of an endoprosthesis where ion implantation is not desired to block (e.g., absorb or deflect) ions. Sacrificial materials include, for example, polymers which absorb noble gas ions without subsequent bubble formation (e.g., a layer of polyurethane or poly(methyl methacrylate) having a thickness more then a couple of micrometers). The sacrificial materials can be removed after ion implantation is completed or can be left on an endoprosthesis.

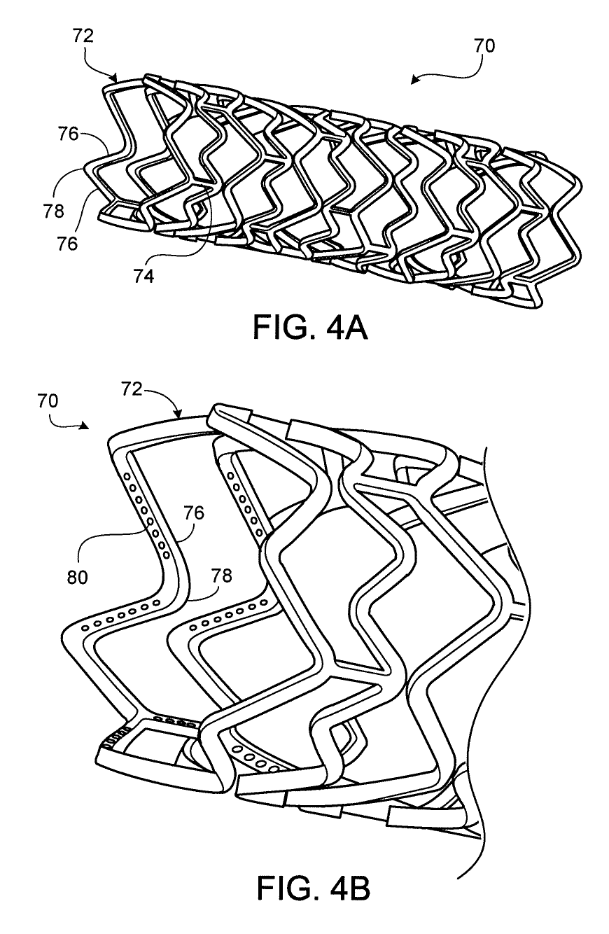

Referring to FIG. 3, methods of making an endoprosthesis 50 can include applying a sacrificial material 52 to a pre-endoprosthesis 54. Sacrificial material 52 can be used to mask portions of pre-endoprosthesis 54 where ion implantation is not desired. Sacrificial material 52 can be applied to face 53 of pre-endoprosthesis 54 upon which ions will be applied. In some embodiments, sacrificial material 52 can be applied along the edges of pre-endoprosthesis 54 and in locations where apertures 56 will be formed in endoprosthesis 50.

Ions of the noble gas can then be accelerated towards face 53 of pre-endoprosthesis 54 thus forming pores 58 as described above with reference to FIGS. 1A, 1B, 2A and 2B. By leaving a buffer around the edges of pre-endoprosthesis 54 and around the locations where apertures 56 will be formed, pores 58 can be formed which open to face 53 but not to end surfaces 60 and aperture surfaces 62 of finished endoprosthesis 50. As described above, pores 58 can be formed with an interior layer whose porosity is greater than the porosity of a surface layer. In some embodiments, a high enough dose of the noble gas ions is applied to pre-endoprosthesis 42 that pores 58 break through face 53. In some embodiments, ion implantation is halted before breakthrough occurs and portions of face 53 are removed (e.g., by chemical etching or ion beam milling) to provide openings to pores 58.

Bores 64 can then be formed (e.g., by ion milling or laser machining) extending from face 53 through the surface layer of pores into the interior layer of larger pores. A therapeutic agent can then be loaded into the interior layer of larger pores. For example, pre-endoprosthesis 54 with pores 58 and bores 64 already formed can be immersed in a liquid pharmaceutical compound for sufficient period of time for the pharmaceutical compound to substantially fill pores 58. In another example, a therapeutic agent can be injected through bores 64 into the interior layer of larger pores. Plugs 66 can then be inserted into bores 64 to limit flow of the therapeutic agent out of the interior layer of larger pores through the bores.

Sacrificial material 52 (e.g., a layer of polyurethane or poly(methyl methacrylate)) can be removed from pre-endoprosthesis 42 before the pre-endoprosthesis is formed into a tubular member. In some embodiments, techniques to remove sacrificial material 52 (e.g., chemical etching or ion beam milling) can be applied after the interior layer of larger pores is loaded with the therapeutic agent. This sequencing can prevent contamination of the pores with, for example, a chemical etchant. In some embodiments, sacrificial material 52 can be removed after pre-endoprosthesis 42 is formed into a tubular member. In some embodiments, sacrificial material 52 can be left on pre-endoprosthesis 42.

Pre-endoprosthesis 42 can then be wound (e.g., circumferentially around a mandrel) and opposing longitudinal edges 68 of the sheet can be joined together, e.g., by welding or by an adhesive, to form tubular member 70. Tubular member 70 can be drawn and/or cut to size, as needed, and portions of the tubular member removed to form apertures 56 of endoprosthesis 50. Endoprosthesis 50 can be cut and/or formed by laser cutting, as described in U.S. Pat. No. 5,780,807, hereby incorporated by reference in its entirety.

Similar methods can be used produce endoprostheses with other configurations. For example, the compression and expansion that occur during installation of an endoprosthesis produce stresses that are typically concentrated at the joints whose bending enables such compression and expansion. As the presence of pores may reduce the strength of portions of endoprostheses where the pores are present, it may be desirable to prevent iron implantation and related pore formation in the vicinity of such joints.

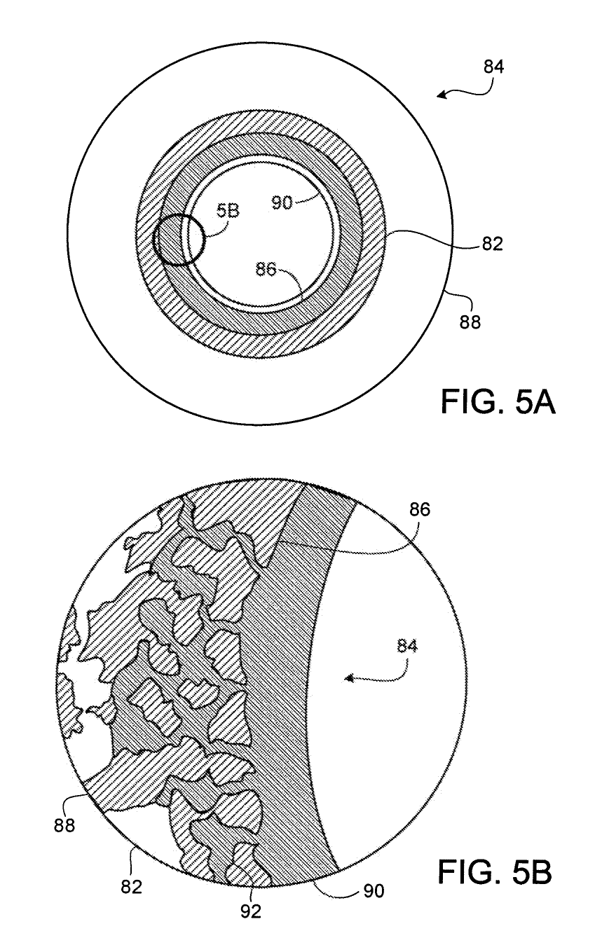

Referring to FIGS. 4A and 4B, methods similar to that described with reference to FIG. 3 can be used to form an endoprosthesis 70 with rings 72 joined together by struts 74. Each ring 72 includes multiple straight members 76 joined together at elbows 78. Stresses created during compression and expansion of endoprosthesis 70 tend to be concentrated at elbows 78. Accordingly, endoprosthesis 70 includes pores 80 located in straight members 76 but not in elbows 78. In other embodiments, masking techniques can be applied to limit pore formation in areas of a medical device or endoprosthesis where structural stability and/or strength are of concern.

In certain embodiments, an endoprosthesis can include a coating that contains a therapeutic agent or that is formed of a therapeutic agent. For example, an endoprosthesis can include a coating that is formed of a polymer and a therapeutic agent. The coating can be applied to a generally tubular member of the endoprosthesis by, for example, dip-coating the generally tubular member in a solution including the polymer and the therapeutic agent. Methods that can be used to apply a coating to a generally tubular member of an endoprosthesis are described, for example, in provisional U.S. Patent Application Ser. No. 60/844,967, filed Sep. 15, 2006 and entitled “Medical Devices”

Examples of coating materials that can be used on an endoprosthesis include metals (e.g., tantalum, gold, platinum), metal oxides (e.g., iridium oxide, titanium oxide, tin oxide), and/or polymers (e.g., SIBS, PBMA). Coatings can be applied to an endoprosthesis using, for example, dip-coating and/or spraying processes.

In addition to being used to form pores in a drug-eluting endoprostheses, ion implantation can be used as a surface treatment technique to prepare metal endoprostheses to receive coatings (e.g., polymeric or ceramic coatings). For example, a metallic endoprosthesis can be coated with a drug bearing polymer on its lumenal surface. The resulting endoprosthesis can provide advantages associated with metallic endoprostheses such as, for example, good strength, structural stability, and biocompatibility as well advantages associated with polymeric or polymer-coated endoprostheses such as, for example, good pharmaceutical compound retention and elution characteristics. However, smooth surfaces of metallic endoprostheses can, in some embodiments, make it difficult to attach such coatings to the endoprostheses. Using ion implantation can form with a surface layer of pores on endoprostheses thus providing attachment points for a coating (e.g., a ceramic or polymeric layer).

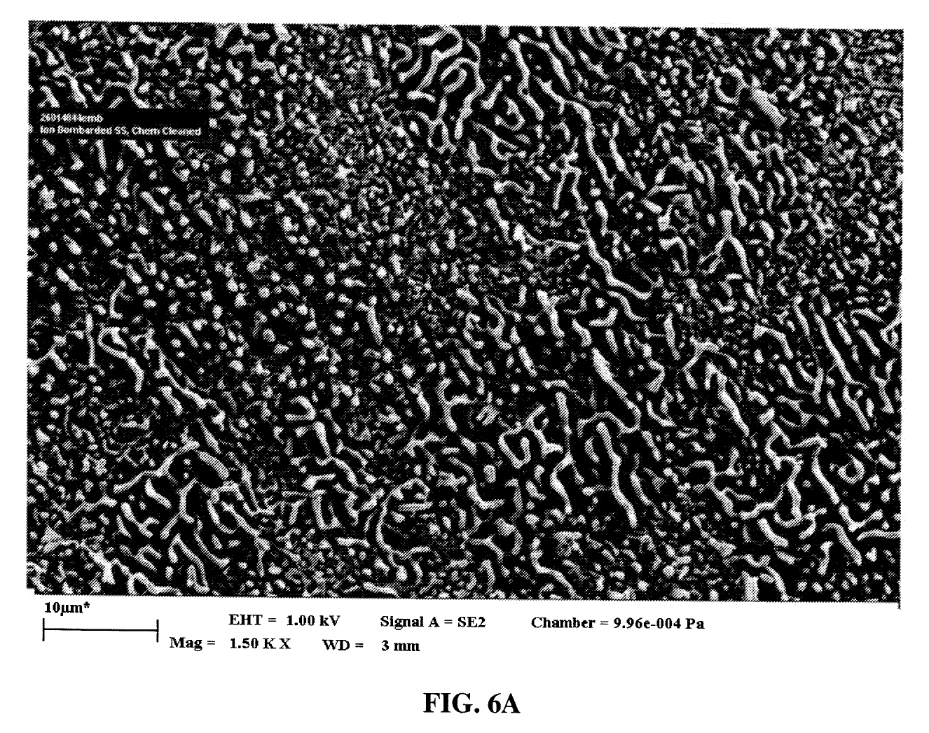

Referring to FIGS. 5A and SB, ion implantation can be used to form pores 82 extending into an endoprosthesis 84 from a lumenal surface of a metal portion 88 of the endoprosthesis. In this embodiment, endoprosthesis 84 also includes a drug-bearing polymeric coating 90 (e.g., styrene-isobutylene styrene (SIBS), polyglycolicacid (PLGA), or polyurethane). Application of polymeric coating 90 in liquid form to portions of the endoprosthesis 84 in which pores 82 have been formed by ion implantation allows the liquid polymer to infiltrate into the pores before setting. Interconnected pores 82, especially interconnected pores which increase in characteristic size with increasing distance from lumenal surface 86, can provide for a strong attachment between metal portion 88 and polymeric coating 90. Polymeric coating 90 can effectively be anchored by solidified portions of the coating which have set in nodes 92 of pores 82 which are larger than channels 94 connecting the nodes to lumenal surface 86.

In some embodiments, pores 82 and polymeric coating 90 are located over substantially the entire lumenal surface 86 of metal portion 88 of endoprosthesis 84. In some embodiments, pores 82 and/or polymeric coating 90 are located in only a portion of lumenal surface 86. In some embodiments, polymeric coating 90 is only applied over portions of lumenal surface 86 where pores 82 are present. In some embodiments, polymeric coating 90 is applied to both portion of lumenal surface 86 where pores 82 are not present and portions of the lumenal surface where the pores are present to act as anchoring points. As discussed above, other coatings including, for example, ceramic coatings, can use pores formed using ion implantation as attachment points in other embodiments of coated endoprostheses.

Pore formation in stainless steel using ion implantation has been investigated through a series of trials using argon and helium ions. In general, these trials used samples of stainless steel that were 12 millimeters by 8 millimeters by 1 millimeter in size. Trial-specific ion implantation parameters are presented in Table 1. Common ion implantation parameters included RF power of 350 Watts, pulse duration of 5 micro seconds, plasma pressure of argon 0.2 pascal, and pressure of helium 0.35 pascal.

TABLE 1

| | | | Dose | | |
|---|---|---|---|---|---|
| Sample | Ions | Eᵢₒₙ (KeV) | (ions/cm²) | Hₚᵤₗₛₑ (Hz) | Tₘₑₐₛ (C.) |
| SS-06A | Ar⁺ | 35 | 50 × 10¹⁷ | 500 | 340 |
| SS-07 | Ar⁺ | 35 | 20 × 10¹⁷ | 800 | 330 |
| SS-08 | Ar⁺ | 35 | 50 × 10¹⁷ | 800 | 420 |
| SS-09 | Ar⁺ | 35 | 20 × 10¹⁷ | 500 | 450 |
| SS-10 | He⁺ | 30 | 20 × 10¹⁷ | 400 | 130 |
| SS-11 | He⁺ | 30 | 50 × 10¹⁷ | 800 | 170 |
| SS-12 | He⁺ | 30 | 50 × 10¹⁷ | 400 | 100 |

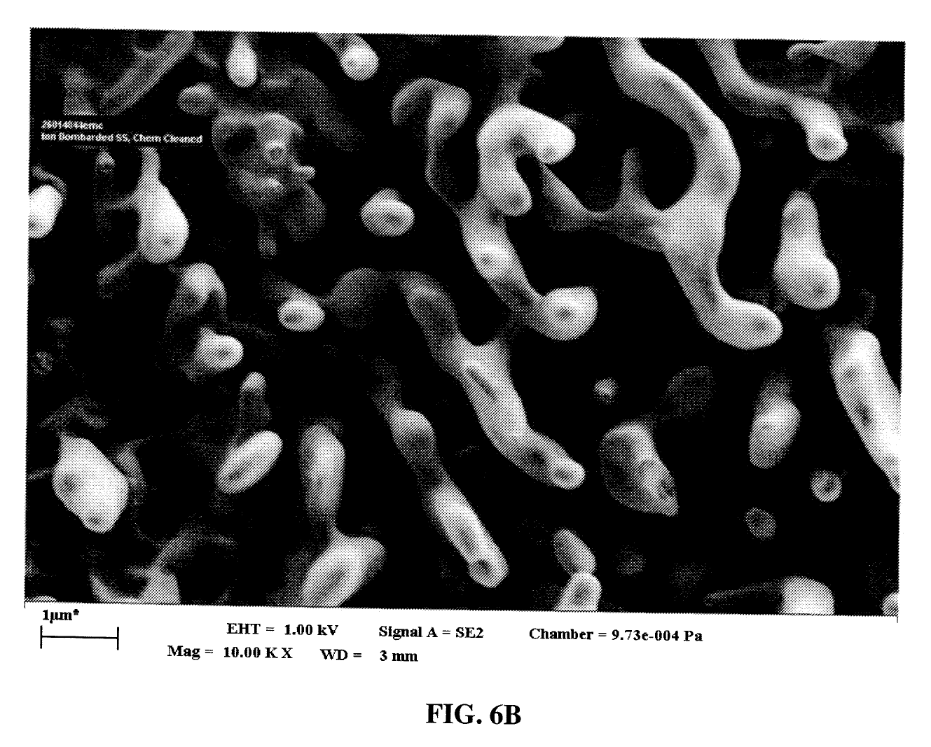

Referring to FIGS. 6A and 6B, scanning electron micrographs taken of a cross-section of a sample at 1,500 and 10,000 magnifications respectively illustrate the pore structures that can be formed using ion implantation. Scales are provided on the lower left portion of each micrograph. The micrograph show voids as light areas and stainless steel portions as dark areas. The shading of the light areas reflects the amount of metal between the cross-section and individual voids and, thus, the distance of individual voids from the cross-section surface. As can be seen here, ion implantation of argon can be used to produce interconnected pores with a representative pore size of about 0.5 micrometers.

A number of embodiments of the invention have been described. Nevertheless, other embodiments are also possible. For example, ion implantation can be used to form pores in other medical devices including, for example, dental implants and bone implants. In some applications (e.g., dental implants), ion implantation parameters can be chosen to for a surface layer of pores with a representative pore size that is smaller than the size of most bacteria (e.g., less than 300 nanometers, 200 nanometers, or 100 nanometers). Such surface pores can provide for the elution of therapeutic agents without providing sanctuaries for bacteria growth.

While endoprostheses including generally tubular members formed out of a metal matrix and/or including a therapeutic agent have been described, in some embodiments, an endoprosthesis can include one or more other materials. The other materials can be used, for example, to enhance the strength and/or structural support of the endoprosthesis. Examples of other materials that can be used in conjunction with a metal matrix in an endoprosthesis include metals (e.g., gold, platinum, niobium, tantalum), metal alloys, and/or polymers (e.g., styrene-isobutylene styrene (SIBS), poly(n-butyl methacrylate) (PBMA)). Examples of metal alloys include cobalt-chromium alloys (e.g., L605), Elgiloy® (a cobalt-chromium-nickel-molybdenum-iron alloy), and niobium-1 Zr alloy. In some embodiments, an endoprosthesis can include a generally tubular member formed out of a porous magnesium matrix, and the pores in the magnesium matrix can be filled with iron compounded with a therapeutic agent.

Accordingly, other embodiments are within the scope of the following claims.

What is claimed is:

1. An endoprosthesis comprising:
a metal member including a porous first portion with pores extending from a surface of the metal member into the first portion and non-porous second portion;
wherein the first portion has a porosity that varies with distance from the surface of the metal member.

2. The endoprosthesis of claim 1, wherein the porosity of first portion increases with distance from the surface.

3. The endoprosthesis of claim 2, wherein the first portion includes a surface layer of pores with a first representative pore size and an interior layer of pores with a second representative pore size that is greater than the first representative pore size, pores of the surface layer interconnected to provide a plurality of fluid flow paths extending between the surface and the interior layer.

4. The endoprosthesis of claim 3, further comprising a therapeutic agent disposed within the interior layer of pores.

5. The endoprosthesis of claim 3, wherein the first representative pore size is between about 0.5 and 5 nanometers.

6. The endoprosthesis of claim 5, wherein the first representative pore size is between about 1.5 and 3 nanometers.

7. The endoprosthesis of claim 3, wherein the second representative pore size is between about 50 nanometers and 500 nanometers.

8. The endoprosthesis of claim 3, further comprising a plug disposed in a bore extending between the surface and the interior layer.

9. The endoprosthesis of claim 2, wherein the metal member is a tubular member having an axis and the first portion is disposed between the second portion and the axis.

10. The endoprosthesis of claim 1, wherein the porous first portion and the non-porous second portion are integrally formed.

11. The endoprosthesis of claim 1, wherein the metal member comprises struts interconnected at junctions and the pores are not present at the junctions.

12. The endoprosthesis of claim 1, further comprising a coating, the coating covering a portion of the surface of the metal member and extending into the pores of the first portion.

13. The endoprosthesis of claim 12, wherein the coating comprises a polymer.

14. The endoprosthesis of claim 12, wherein the coating comprises a ceramic.

15. A medical device comprising:
a metal member including a porous first portion with pores extending from a surface of the metal member into the first portion and non-porous second portion;
wherein the first portion has a porosity that varies with distance from the surface of the metal member.

16. The medical device of claim 15, wherein the porosity of first portion increases with distance from the surface.

17. The medical device of claim 16, wherein the first portion includes a surface layer of pores with a first representative pore size and an interior layer of pores with a second representative pore size that is greater than the first representative pore size, pores of the surface layer interconnected to provide a plurality of fluid flow paths extending between the surface and the interior layer.

18. The medical device of claim 17, further comprising a therapeutic agent disposed within the interior layer of pores.

19. The medical device of claim 17, further comprising a plug filling a bore extending between the surface and the interior layer.

20. The medical device of claim 15, further comprising a coating, the coating covering a portion of the surface of the metal member and extending into the pores of the first portion.

21. The medical device of claim 15, wherein the medical device forms at least part of a dental implant.

22. The medical device of claim 21, wherein the first portion includes a surface layer of pores with a first representative pore size and the first representative pore size is less than about 200 nanometers.

23. The medical device of claim 15, wherein the medical device forms at least part of a bone implant.

24. The medical device of claim 15, wherein the medical device forms at least part of an embolic coil.

25. A method of forming an endoprosthesis, the method comprising:
forming a pre-endoprosthesis from a metal; and
forming pores in the metal by implanting ions of a noble gas in the metal.

26. The method of claim 25, wherein forming the endoprosthesis takes place before forming the pores.

27. The method of claim 25, wherein forming the pores takes place before forming the endoprosthesis.

28. The method of claim 25, wherein the noble gas is selected from the group consisting of argon and helium.

29. The method of claim 25, wherein the metal is selected from the group consisting of titanium, stainless steel, stainless steel alloy, tungsten, tantalum, niobium, and zirconium.

30. The method of claim 25, further comprising covering portions of the metal with a sacrificial material which limits ion implantation.

31. The method of claim 30, further comprising removing the sacrificial layer.

32. The method of claim 25, wherein implanting the ions comprises applying the ions at an implantation energy of between about 10 kiloelectronvolts and 1 megaelectronvolts.

33. The method of claim 25, wherein implanting the ions comprises applying the ions at a dose of between about 15×10¹⁷ and 50×10¹⁸ ions per square centimeter.

34. The method of claim 25, forming the pores comprises forming a surface layer of pores with a first representative pore size and an interior layer of pores with a second representative pore size that is greater than the first representative pore size, pores of the surface layer interconnected to provide a plurality of fluid flow paths extending between a surface of the metal and the interior layer of pores.

35. The method of claim 34, further comprises:
forming a bore extending from the surface of the metal to the interior layer of pores;
loading a therapeutic agent into the interior layer of pores; and
placing a seal material in the bore.

36. The method of claim 25, further comprising applying a mask to control locations at which pores are formed in the metal.
